(19) United States
(12) Patent Application Publication
(10) Pub. No.: US 2019/0015588 A1
(43) Pub. Date: Jan. 17, 2019

(54) INLET TUBE SET FOR SOURCE INGREDIENT DELIVERY

(71) Applicant: BAXTER CORPORATION ENGLEWOOD, Englewood, CO (US)

(72) Inventors: Brian William WARD, Littleton, CO (US); Cari Lyn HEFFNER, Castle Rock, CO (US); Megan Marie KLECKNER, Denver, CO (US)

(73) Assignee: BAXTER CORPORATION ENGLEWOOD, Englewood, CO (US)

(21) Appl. No.: 16/067,195

(22) PCT Filed: Dec. 22, 2016
(86) PCT No.: PCT/US16/68234

§ 371 Date: Jun. 29, 2018

Related U.S. Application Data
(60) Provisional application No. 62/272,816, filed on Dec. 30, 2015.

(51) Int. Cl. A61M 5/162; A61M 5/32

(52) U.S. Cl. CPC A61M 5/1626; A61M 5/50; A61M 5/3213

(57) ABSTRACT

A medical tube set is provided, including a length of tubing having a first end, and an opposite second end, the first end connected to a spike. An outlet valve is connected to the second end and forms a connector configured for affixing the second end to a medication delivery device, the connector having a connector body having an inlet end and an opposite delivery end. A tip cover is slidably engaged on the delivery end and is movable between an extended position in which the cover obscures the delivery end, and a retracted position in which the delivery end is exposed for delivery of fluid from the tubing in a medical apparatus.

### RELATED APPLICATION

This application claims priority to U.S. Patent Application Ser. No. 62/272,816, filed on Dec. 30, 2015, entitled “INLET TUBE SET FOR SOURCE INGREDIENT DELIVERY”, which application is incorporated herein by reference in its entirety. This application relates to and incorporates by reference the co-owned U.S. Patent Application Ser. No. 62/272,786, filed on Dec. 30, 2015, entitled “SYRINGE POSITIONING APPARATUS AND METHOD”. This application relates to and incorporates by reference the co-owned U.S. Patent Application No. 62/272,789, filed on Dec. 30, 2015, entitled “MEASUREMENT OF SYRINGE GRADUATION MARKS USING A VISION SYSTEM”. This application relates to and incorporates by reference the co-owned application U.S. Patent Application No. 62/272,794, filed on Dec. 30, 2015, entitled “CAPACITIVE SINGLE PLATE BUBBLE DETECTOR”. This application relates to and incorporates by reference the co-owned U.S. patent application Ser. No. 14/984,022, filed on Dec. 30, 2015, entitled “SOURCE FLUID INLET ASSEMBLY FOR AUTOMATED FILLING DEVICE”. This application relates to and incorporates by reference the co-owned U.S. Patent Application No. 62/272,798, filed on Dec. 30, 2015, entitled “SYRINGE GRIPPING APPARATUS AND METHOD”. This application relates to and incorporates by reference the co-owned U.S. patent application Ser. No. 14/984,285, filed on Dec. 30, 2015, entitled “SYRINGE PLUNGER POSITION APPARATUS AND METHOD”. This application relates to and incorporates by reference the co-owned U.S. patent application Ser. No. 14/984,913, filed on Dec. 30, 2015, entitled “TIP CAP FOR AUTOMATIC SYRINGE FILING APPARATUS”. This application relates to and incorporates by reference the co-owned U.S. patent application Ser. No. 15/179,643, filed on Jun. 10, 2016, entitled “TAMPER EVIDENT CAP”. This application relates to and incorporates by reference the co-owned U.S. patent application Ser. No. 15/360,635, filed Nov. 23, 2016, entitled “LABEL APPLICATOR FOR SYRINGE LABELING”.

### BACKGROUND

The present invention relates to systems for formulating and/or delivering medication to patients through use of an automatic filling/compounding device, and more specifically to a tube set having a connection end that reduces touch contamination and facilitates the transfer of accurate amounts of fluid to containers being processed by the automatic filling/compounding device.

Medical tube sets are well known in the art, and typically include a long, transparent or translucent flexible plastic tube having a spike at one end for placing the tube in fluid communication with a container, such as a bag, of the desired medical solution. Such solutions include saline, medicines, components of medicines which must be placed in a second container prior to delivery of the solution to the patient, and the like. The containers are often mounted to a rack to suspend them such that the liquid contents of the container are available to be transferred to a desired second container, such as a syringe, a patient specific container or other medical container.

Opposite the spike end, the length of tubing is provided with a connector for achieving fluid communication between the suspended container discussed above, and the desired second container. The second container may be a container being filled by an automatic filling/compounding device such as an automatic syringe filling apparatus or other medical devices or systems known in the art and the first container may be a source solution container.

A common problem of such systems is that when the nurse or medical technician operably attaches the connector to the automatic filling/compounding device, special care needs to be taken to avoid contaminating the end of the connector which may then come into contact with the fluid being transferred, which is called “touch contamination.” Another problem of conventional tube sets is that the volume of solution delivered from the suspended container to the second container must be accurately measured, and therefore voids or bubbles should be detected so appropriate measures may be taken. A further problem is that the tube sets for each or a plurality of source solution containers are not adequately distinguished from each other such that the source solution being transferred to the second container may be misidentified as another available source solution.

Accordingly, there is a need for an improved tube set that addresses the above-listed drawbacks of conventional technology.

### SUMMARY

The above-listed needs are met or exceeded by the present inlet tube set for source ingredient delivery, which features a disposable tube set having an integrated luer activated valve (LAV) that includes features for preventing touch contamination, has a unique device identifier (UDI), preferably a barcode label, for identification and counterfeit protection, provides for bubble detection, and is mechanically locked into a filling device when in use. The present tube set is connectable to a source container of IV fluid, saline, or other medication or medicinal solution, and is preferably insertable into an automated syringe filling, compounding or other device for adding fluid to a second container. For the purposes of the present application, such devices are collectively referred to as a “medication delivery device.”

A retractable tip cover reduces the likelihood of touch contamination of a critical exposed portion of the LAV that comes into contact with the fluid being transferred when the LAV is inserted into the automated device by shielding the critical contact surfaces until the LAV is inserted into the medication delivery device. A feature of the present retractable tip cover is that it only retracts to expose the LAV at the final stage of the insertion into the medication delivery device. Contact with complementary formations in the medication delivery device cause the axial retraction of the tip cover from a default, extended or covering position, to a retracted position exposing the contact surface of the LAV.

The UDI allows the user to associate the device with the source ingredient, track the duration of use and number of valve actuations, and verify the authenticity of the disposable. A window positions the tubing in communication with a sensor on the filling device for bubble detection. In addition, the configuration of the present tube set includes a connector configured for interacting with complementary features on the medication delivery device for preventing the user from removing the tube set from the medication delivery device during use. More specifically, the present invention provides a medical tube set, including a length of tubing having a first end, and an opposite second end, the first end connected to a spike. An outlet valve is connected to the second end and forms a connector configured for affixing the second end to a medication delivery device, the connector including a connector body having an inlet end and an opposite delivery end. A tip cover is slidably engaged on the delivery end and is movable between an extended position in which the cover obscures the delivery end, and a retracted position in which the delivery end is exposed for delivery of fluid from the tubing in a medical apparatus.

In another embodiment, an outlet valve is provided for a medical tube set including a length of tubing constructed and arranged for connecting a container of medicinal solution to a medication delivery device, and includes a connector including a connector body having an inlet end and an opposite delivery end, a tip cover slidably engaged on the delivery end and movable between an extended position in which the cover completely obscures the delivery end, and a retracted position in which the delivery end is exposed for delivery of fluid from the tubing into the medication delivery device. A bubble detection window portion is located between the inlet end and the delivery end, and a clip is associated with the inlet end for releasably locking the valve to the medication delivery device.

Various embodiments may comprise any number of combinations of apparatus and/or method features described above and/or hereinbelow. Such combinations may include those encompassed by the following Embodiments:

1. A medical tube set, comprising:

a length of tubing having a first end, and an opposite second end, said first end connected to a spike;

an outlet valve connected to said second end and forms a connector configured for affixing said second end to a medication delivery device, said connector including a connector body having an inlet end and an opposite delivery end;

a tip cover slidably engaged on said delivery end and movable between an extended position in which said cover obscures said delivery end, and a retracted position in which said delivery end is exposed for delivery of fluid from said tubing in a medical apparatus.

2. The tube set of Embodiment 1, wherein said tip cover includes petals that open when said outlet valve is connected to the medication delivery device and said tip cover is moved to said retracted position.

3. The tube set of Embodiment 1 or Embodiment 2, wherein said connector body includes an integral bubble detection window portion.

4. The tube set of any one of Embodiments 1-3, wherein said bubble detection window portion has a generally planar platform and includes a bubble detection window generally axially positioned on said platform.

5. The tube set of any one of Embodiments 1-4 wherein said bubble detection window portion is located between said inlet end and said delivery end.

6. The tube set of any one of Embodiments 1-5, wherein said inlet end is provided with a clip for releasably locking said valve to the medication delivery device.

7. The tube set of any one of Embodiments 1-6, wherein said inlet end is provided with a unique device identifier for the tube set.

8. The tube set of any one of Embodiments 1-7, wherein said unique device identifier is a barcode label.

9. An outlet valve for a medical tube set, comprising:

a connector including a connector body having an inlet end and an opposite delivery end;

a tip cover slidably engaged on said delivery end and movable between an extended position in which said cover obscures said delivery end, and a retracted position in which said delivery end is exposed for delivery of fluid from said tubing in a medical apparatus;

a bubble detection window portion located between said inlet end and said delivery end; and

a clip associated with said inlet end for releasably with a clip for releasably locking said valve to the medication delivery device.

10. The outlet valve of Embodiment 9, wherein said bubble detection window portion has a generally planar platform and includes a bubble detection window generally axially positioned on said platform.

11. The outlet valve of Embodiment 9 or Embodiment 10, wherein said inlet end is provided with a unique device identifier for the tube set.

12. The outlet valve of any one of Embodiments 9-11, wherein said unique device identifier is a barcode label.

13. The outlet valve of any one of Embodiments 9-12, wherein said connector body has at least one locking lug for retaining said tip cover in at least one of said extended and retracted positions.

14. The outlet valve of any one of Embodiments 9-13, wherein said tip cover includes a barrel with at least one radially projecting guide rib.

Additional features and advantages of the present invention will become apparent upon consideration of the description that follows.

### BRIEF DESCRIPTION OF THE DRAWINGS

FIG. 1 is a front elevation of the present tube set;

FIG. 2 is a top perspective view of the present connector showing the cover obscuring the connection end;

FIG. 3 is a top perspective view of the connector of FIG. 2 showing the cover retracted, exposing the connection end;

FIG. 4 is a top perspective view of the present retractable tip;

FIG. 5 is an opposite side top perspective view of the tip of FIG. 4;

FIG. 6 is a rear perspective view of the tip of FIG. 4;

FIG. 7 is a bottom perspective view of the connector of FIGS. 2 and 3;

FIG. 8 is a fragmentary vertical cross-section of the present tube end secured in a portion of an automatic loading/filling machine; and

FIG. 9 is a fragmentary bottom perspective view of the present tube end secured in the portion of the automatic loading/filling machine, and disclosing the bar code scanner.

### DETAILED DESCRIPTION

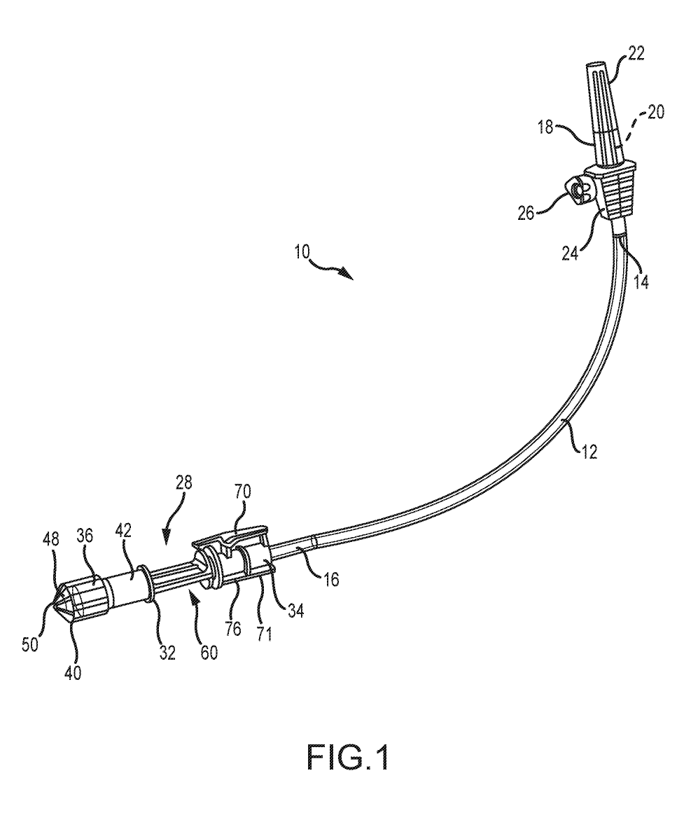

Referring now to FIG. 1, the present medical tube set is generally designated 10 and includes a length of tubing 12 having a first end 14 and an opposite second end portion 16. As is known in the art, the tubing 12 is preferably made of flexible plastic, and is also preferably transparent. However, other materials are contemplated, depending on the particular application or medicinal solution to be delivered. Also, tubing made of translucent, or opaque materials is also contemplated. The first end 14 is connected to a conventional spike 18, needle or other known connector, herein referred to as a spike, constructed and arranged with a pointed end 20, here shown covered by a sheath 22. As is known in the art, the pointed end 20 is constructed and arranged for puncturing an outlet port (not shown) found on a container of medical solution, typically a flexible plastic bag, often suspended from a rack to promote flow of the solution into the tubing 12. A spike body 24 is in fluid communication with the pointed end 20 and is provided with a stop-cock valve 26 rotatable by an operator, such as a nurse or medical technician, for controlling flow from the container into the tubing 12.

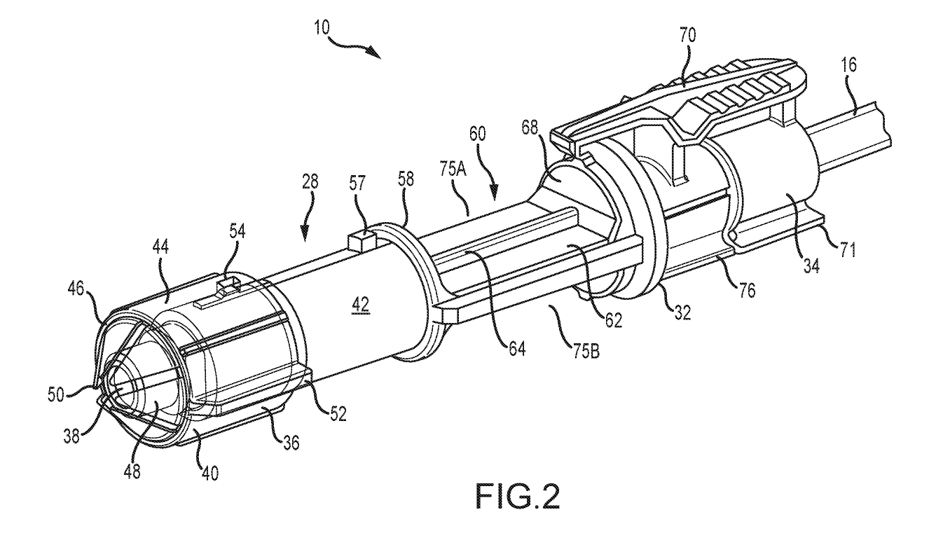

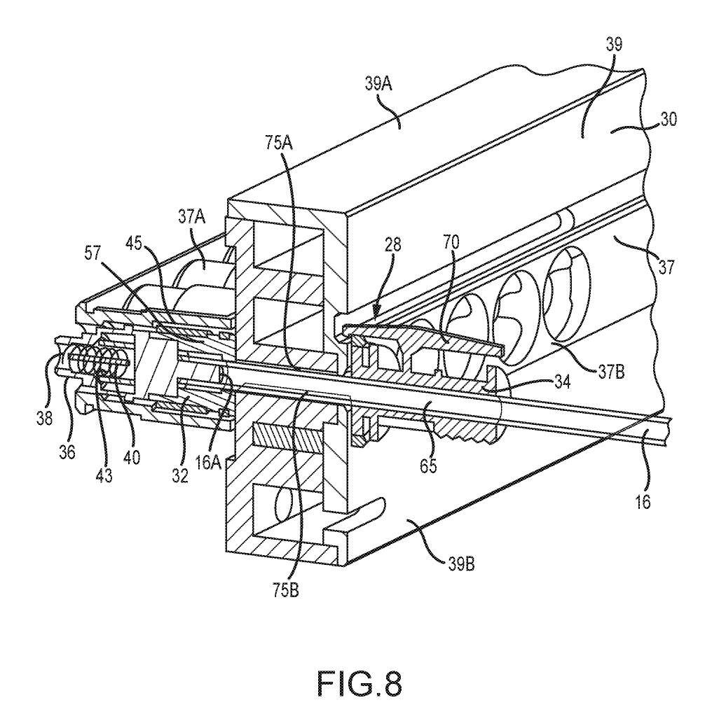

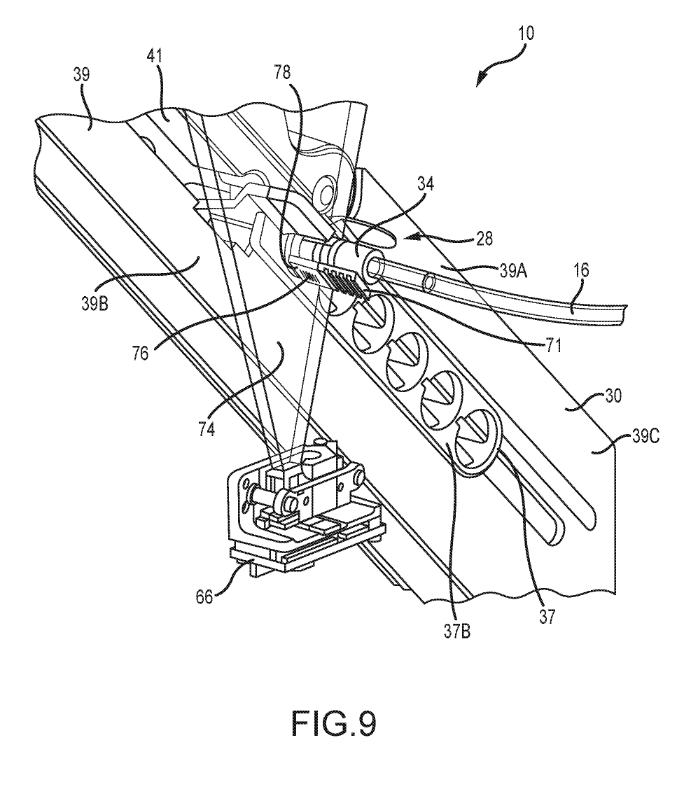

Referring now to FIGS. 2, 3 and 7, opposite the spike 18, at the second end portion 16 is provided an outlet valve 28 forming a connector configured for affixing the second end portion to a medication inlet assembly 30 (FIGS. 8 and 9) forming a component of a medication delivery device and in an embodiment the device is an automatic syringe loading/filling machine. The valve 28 includes a connector body 32 having an inlet end 34 and an opposite outlet or delivery end 36.

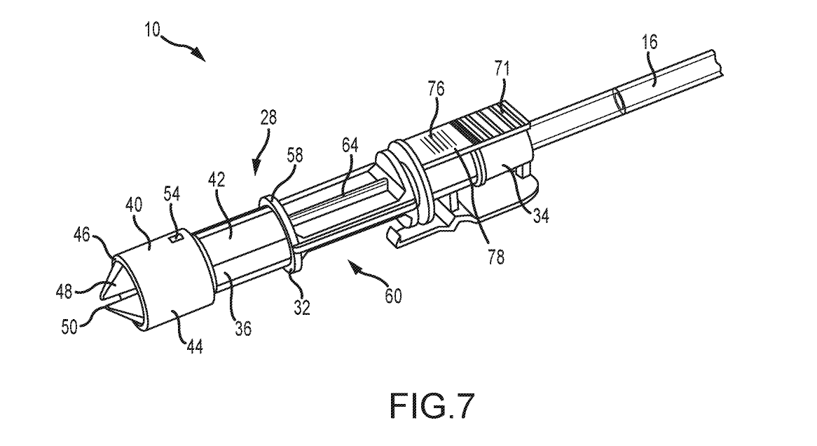

Referring to FIGS. 8 and 9, the inlet mount or inlet assembly 30 includes a cartridge 37 which includes a forward section 37A formed to slidingly accommodate the delivery end 36 of the valve 28 and a rearward flange 37B. The inlet assembly 30 also includes a track 39 having an first or upper track member 39A that moves relative to a second or lower track member 39B from an open position to allow for insertion of the cartridge 37 to a clamped position to form a slot 41 extending therebetween to accommodate and clamp a suitably configured portion of the cartridge 37 therebetween. As seen in FIG. 9, the slot 41 optionally extends into a portion 39C of the track 39 in which the upper and lower sections are formed in a fixed relationship relative to each other. The track 39 is operatively attached to an apparatus (not shown) that provides for reciprocal lateral movement (when viewed along the length of the track 39) of the cartridge 37 within the slot 41 to place the inlet mount in desired positions. Such positions including a position for allowing insertion of the connector body 32 into the clamped cartridge 37, a position allowing for bubble detection, and a position for allowing scanning of a label 78 (FIG. 7) as is described below. Two or more of these various positions may be the same position.

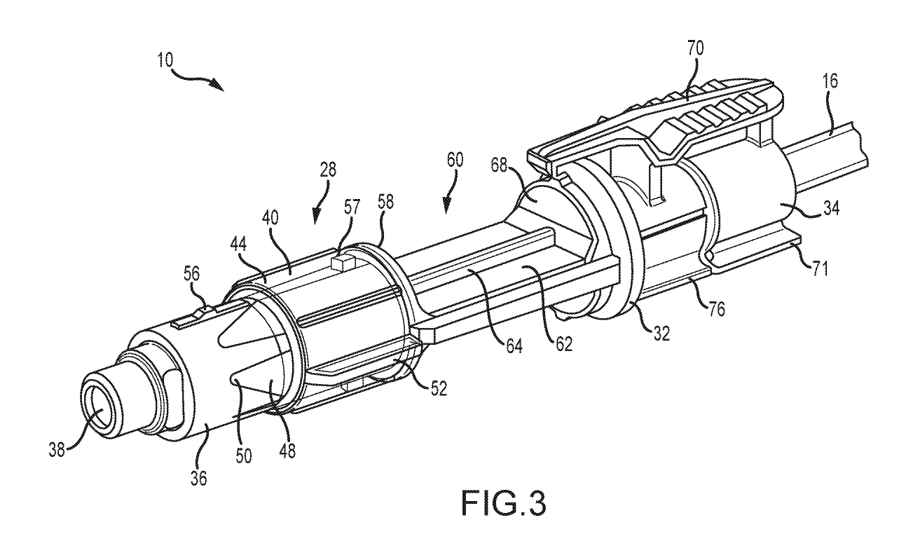

Referring back to FIGS. 2, 3 and 7, a tip cover 40 is slidably engaged on the delivery end 36 and is axially movable relative to the connector body 32 between an extended position (FIG. 2) in which the cover obscures the delivery end 36, and a retracted position (FIG. 3) in which the delivery end is exposed for delivery of fluid from the tubing 12 into containers (not shown) being processed through the automatic loading/filling machine. The delivery end 36 is preferably axially long enough to define a neck portion 42 dimensioned for accommodating the tip cover 40 in the retracted position.

As is known in the art, the delivery end 36 is preferably formed as a Luer Activated Valve (LAV) which has a deformable internal element 43 (FIG. 8) that is in a default closed position, but is deformed into an open position upon engagement of the LAV to a suitable connector fitting (not shown) to create fluid communication to containers being processed by the automatic loading/filling machine. Upon disengagement of the connector fitting, the internal element 43 recovers back to the closed position. A tip 38 (FIG. 3) of the internal element 43 contacts the connector fitting upon the inserting engagement of the connector fitting to the LAV, and the contact deforms the internal element 43 into the open position. As the tip 38 may come into contact with fluid being transferred to the containers, to maintain sterility it is important to avoid touch or other contact of the tip 38 by anything prior to connection to the connector fitting. As is best shown in FIG. 8, in an embodiment, the delivery end 36 includes a base 45 that is directly attached to an outlet end 16a of the outlet end portion 16 of the tubing 12 to provide sealed, fluid communication between a passageway in the tubing 12 and the LAV.

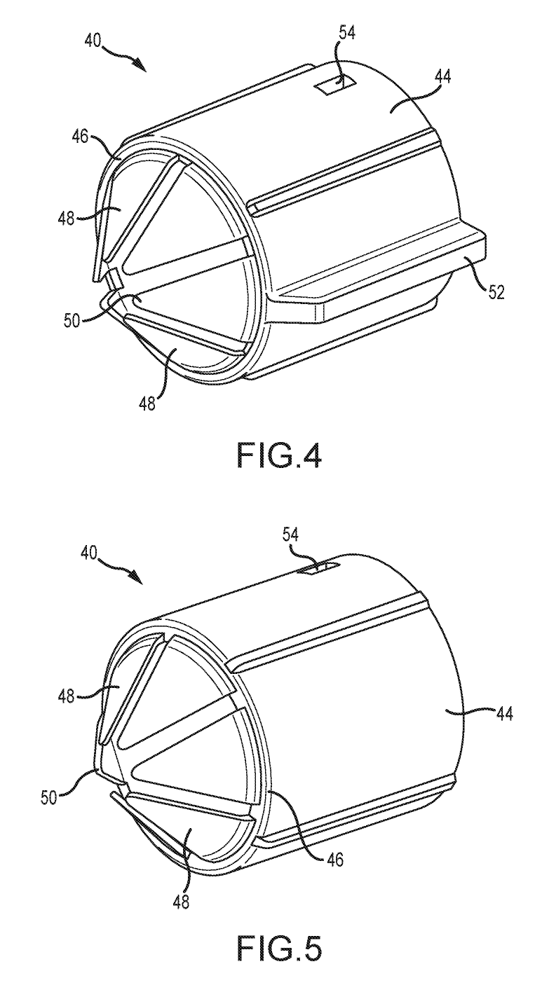

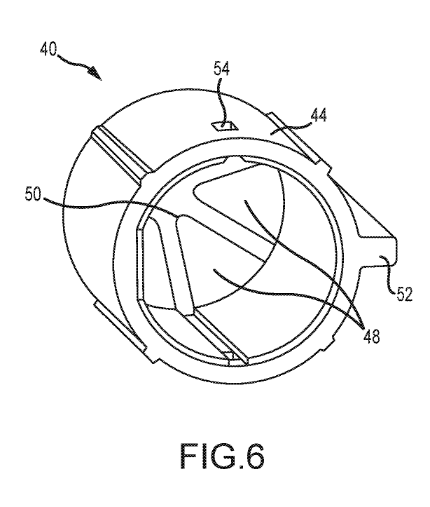

Referring now to FIGS. 2-6, the tip cover 40 includes a generally cylindrical, hollow barrel 44 having a barrel outlet 46 from which extend a plurality of flexible petals 48. The barrel 44 is dimensioned to slidably engage the outlet end 36 for axial movement between the extended position (FIG. 2) and the retracted position (FIG. 3). In an embodiment, the tip cover 40 is formed of a polymeric material such as polypropylene. It is also envisioned that the tip cover 40 can be perforated, frangible membrane, foil or thin film. In an embodiment, the petals 48 are integrally formed from the barrel 44 and have pointed tips 50 that, in a default extended position, cover the outlet tip 38 of the delivery end 36 of the outlet valve 28 and prevent touch contamination while the valve is handled by medical personnel, whether by contact with the medical personnel or by contacting a portion of the inlet assembly 30 such as the cartridge 37 (FIG. 8) upon insertion therein. Also, as seen in FIGS. 4-6, the petals 48 are peripherally spaced from each other to facilitate flexing as the connector is placed into the inlet assembly 30. The petals 48 open as the tip cover 40 moves axially to the retracted position. Alternately, the petals 48 are made of a frangible material created by material thickness or perforations that separate to open.

Another feature of the tip cover 40 is at least one axially extending, radially projecting guide rib 52 constructed and arranged to engage and be guided rearwardly by complementary formations in the forward section 37A of the cartridge 37. It is contemplated that the number and orientation of the ribs 52 may vary to suit the application. Multiple ribs 52 are shown in FIGS. 4-6. Yet another feature of the tip cover 40 is a retaining aperture 54 on the barrel 44 that accommodates a locking lug 56 (FIG. 3) projecting radially from the delivery end 36. The engagement of the lug 56 in the retaining aperture 54 holds the tip cover 40 in position with the petals 48 covering the delivery end 36 until the valve 28 is placed into the medication delivery device 30. As seen in FIG. 3, the neck portion 42 is optionally fitted with a neck lug 57 that holds the barrel 44 in the retracted position.

An annular ring 58 projects radially from the neck portion 42 to define a rearward stop for the barrel 44 as it reaches the retracted position, and also to define a transition between the delivery end and a bubble detection window portion 60.

As seen in FIGS. 2 and 3, the bubble detection window or neck portion 60 is located between the inlet end 34 and the outlet end 36 of the connector body 32. In the preferred embodiment, the bubble detection window or neck portion 60 is integral with the connector body 32, has a generally planar platform 62 and includes a bubble detection window 64 generally axially positioned on the platform. The window 64 is preferably transparent or translucent, is tubular and forms part of a passageway 65 (FIG. 8). As best shown in FIG. 8, in an embodiment, the window 64 is formed by the end portion 16 of the tubing 12 and the planar platform 62 forms a fixture to properly retain and position the end portion 16. In other embodiments, the window 64 is optionally formed as a generally cylindrical tubing separate from the tubing 12 and attached to the end 16A of the tubing 12 and the base 45. Further, the window 64 is disposed on the connector body 32 so that the window is movable into a desired position relative to a suitable sensor (not shown) on the medication delivery device 30 that detects the presence of bubbles in the liquid flowing in the passageway 65 (FIG. 8). The bubble sensor is contemplated as being capacitive, ultrasonic, optical or the like.

Referring again to FIGS. 2 and 3, the rearward ring 68 projects radially from the connector body 32 and defines a transition between the bubble detection window portion 60 and the inlet end 34 of the connector body 32. A main feature of the inlet end 34 is a user releasable, rocking locking clip 70 affixed to the inlet end and disposed to engage the rearward flange 37b on the cartridge 37 (FIG. 8). Referring to FIGS. 2, 3 and 7, opposite the locking clip 70 is a finger grip 71.

Referring to FIGS. 2, 3 and 7-8, as the outlet valve 28 is slidingly inserted and disposed in the cartridge 37, the tip cover 40 axially retracts to the retracted position through engagement of the tip cover on complementary formations in the forward section 37A of the cartridge, exposing the outlet end 36. Prior to such engagement, the tip cover 40 obscures the outlet tip 38, thereby preventing contact by the medical personnel and/or inlet mount 30. Upon full insertion of outlet valve 28 into the cartridge 37, the clip 70 lockingly engages the rearward flange 37B, holding the valve 28 in position and also providing a tactile and aural indication that the outlet valve has been fully inserted into the cartridge.

Referring to FIGS. 2 and 8-9, the bubble detection window or neck portion 60 formed with the generally planar platform 62 and the axially positioned window 64 bounded by the annular ring 58 and the rearward ring 68, form two “U”-shaped groove or recess configurations 75A, 75B extending in opposite generally normal directions from the planar configured surface of the platform 62 and that provide for the connector body to be retained between the upper track portion 39A and lower track portion 39B of the track 39. The generally planar configuration of the window portion 60 allows the window portion to fit within the slot 41 formed between the upper track portion 39A and lower track portion 39B and allow for lateral reciprocal movement therein contemporaneously with the cartridge 37 into which the connector body 32 has been inserted.

Referring now to FIGS. 7 and 9, another feature of the inlet end 34 of the connector body 32 is a Unique Device Identifier (UDI) mount 76. Preferably a generally flat surface, the UDI mount 76 provides a location for a label 78 for a barcode, RF chip or the like for more positively identifying the set 10 as well as the medical solution to be delivered, and/or the particular patient receiving the medical solution. As seen in FIG. 9, a sensor 66 sends a detector beam 74 that scans the label. The label 78 allows the user to associate the disposable tubing set 10 with the source ingredient, track the duration of use and number of valve actuations, and verify the authenticity of the disposable tubing set 10. In the preferred embodiment, the UDI mount 76 is opposite the locking clip 70 on the inlet end 34. However, it is contemplated that these components could be arranged in other positions on the inlet end.

While a particular embodiment of the present inlet tube set for source ingredient delivery has been described herein, it will be appreciated by those skilled in the art that changes and modifications may be made thereto without departing from the invention in its broader aspects and as set forth in the following claims.

1. A medical tube set, comprising:
a length of tubing having a first end, and an opposite second end, the first end connected to a spike;
an outlet valve connected to the second end and forming a connector configured for affixing the second end to a medication delivery device, the connector including a connector body having an inlet end and an opposite delivery end;
a tip cover slidably engaged on the delivery end and movable between an extended position in which the cover obscures the delivery end, and a retracted position in which the delivery end is exposed for delivery of fluid from the tubing in a medical apparatus.

2. The tube set of claim 1, wherein the tip cover includes petals that open when the outlet valve is connected to the medication delivery device and the tip cover is moved to the retracted position.

3. The tube set of claim 1, wherein the connector body includes an integral bubble detection window portion.

4. The tube set of claim 3, wherein the bubble detection window portion has a generally planar platform and includes a bubble detection window generally axially positioned on the platform.

5. The tube set of claim 3 wherein the bubble detection window portion is located between the inlet end and the delivery end.

6. The tube set of claim 1, wherein the inlet end is provided with a clip for releasably locking the valve to the medication delivery device.

7. The tube set of claim 1, wherein the inlet end is provided with a unique device identifier for the tube set.

8. The tube set of claim 7, wherein the unique device identifier is a barcode label.

9. An outlet valve for a medical tube set, comprising:
a connector including a connector body having an inlet end and an opposite delivery end; a tip cover slidably engaged on the delivery end and movable between an extended position in which the cover obscures the delivery end, and a retracted position in which the delivery end is exposed for delivery of fluid from the tubing in a medical apparatus;
a bubble detection window portion located between the inlet end and the delivery end; and a clip associated with the inlet end for releasably with a clip for releasably locking the valve to the medication delivery device.

10. The outlet valve of claim 9, wherein the bubble detection window portion has a generally planar platform and includes a bubble detection window generally axially positioned on the platform.

11. The outlet valve of claim 9, wherein the inlet end is provided with a unique device identifier for the tube set.

12. The outlet valve of claim 11, wherein the unique device identifier is a barcode label.

13. The outlet valve of claim 9, wherein the connector body has at least one locking lug for retaining the tip cover in at least one of the extended and retracted positions.

14. The outlet valve of claim 9, wherein the tip cover includes a barrel with at least one radially projecting guide rib.
